(19) United States
(12) Patent Application Publication
(10) Pub. No.: US 2006/0211778 A1
(43) Pub. Date: Sep. 21, 2006

(54) Solid catalyst, reaction vessel, and process for producing methanol

(76) Inventors: Kenichi Aika, Kanagawa (JP); Hidenobu Kobayashi, Kanagawa (JP)

Correspondence Address: Willam S Frommer;Frommer Lawrence & Haug, 745 Fifth Avenue, New York, NY, 10151, US

(21) Appl. No.: 10/548,770

(22) PCT Filed: Mar. 10, 2004
(86) PCT No.: PCT/JP04/03153

§ 371 Date: Apr. 7, 2006

(30) Foreign Application Priority Data
Mar. 10, 2003 (JP) 2003-063955

(51) Int. Cl. B01J 23/72; C07C 27/06

(52) U.S. Cl. 518/716; 502/345

(57) ABSTRACT

The present invention provides a solid catalyst with which to synthesize liquid-phase methanol from carbon monoxide and hydrogen which are under pressure, in a solution such as methanol, without executing separation or recirculation. A solid catalyst, in which an ion-exchange resin anion-exchanged to be an alcoxide type is combined with a transition metal such as a Raney type, powder type or supported type, is used for synthesizing methanol from carbon monoxide and hydrogen in a solution such as methanol.

### TECHNICAL FIELD

The present invention relates to a solid catalyst with which to synthesize methanol from carbon monoxide and hydrogen which are under pressure. Further, the present invention relates to a reactor containing the solid catalyst. Further, the present invention relates to a method of producing methanol in which methanol is synthesized in the presence of the solid catalyst.

### BACKGROUND ART

Since methanol burns at low temperatures, it is hoped that methanol will serve as an eco-friendly fuel which does not emit nitrogen oxide, serve as the hydrogen source for fuel batteries and serve as the raw material for chemical products. With the advancement of fuel battery technology, demand for methanol is expected to increase in the future. It is BASF Ltd. that has succeeded in synthesizing methanol for the first time; in 1913, water gas was made to react on a metal oxide at a high temperature and under high pressure, and so methanol was obtained as a mixed alcohol. After that, methanol was synthesized using a zinc oxide-chrome oxide based catalyst on the condition of 623 K and 25-35 MPa by Mittash et al. in 1923, and BASF Ltd. constructed a methanol plant of 3,000 ty⁻¹. This catalytic system was excellent in heat resistance, but required such a high-temperature and high-pressure condition as 573-673 K and 15-20 MPa. In 1966, switching was performed from raw material gas to natural gas which contains less sulfur than water gas of coal coke, and ICI PLC began using a copper-zinc oxide based catalyst capable of reaction at 423-473 K and 5 MPa. Since then, Cu/ZnO/Cr₂O₃ and Cu—ZnO/Al₂O₃ have become the main catalysts for methanol gas-phase synthesis. Methanol is synthesized on condition of 503-600 K in reaction temperature, 5-20 MPa in pressure and 10,000-40,000 h⁻¹ in space velocity, and a methanol space time yield of 0.5-4.8 kgL⁻¹h⁻¹ is obtained per catalytic layer volume.

Methanol synthesis from carbon monoxide and hydrogen is an exothermic reaction involving a large decrease in the number of molecules, and becomes more advantageous as the temperature becomes low and the pressure becomes high (Formula 1-1). As regards gas-phase synthesis requiring a high temperature, from the viewpoint of reaction rate, a high conversion cannot be obtained in equilibrium, and the massive accumulation of reaction heat needs to be avoided, so that a 1 pass conversion is restricted, and unconverted gas is recycled. In order to synthesize methanol highly efficiently, what has to be achieved is to gain a sufficient reaction rate at a low temperature and efficiently remove reaction heat generated. Accordingly, much attention has been paid to low-temperature liquid-phase methanol synthesis.

Formula 1
CO+2H₂→CH₃OH(g) [symbol] H°₂₉₈=−90.97 kJmol⁻¹ (1-1)

In a low-temperature liquid-phase synthesis process, reaction heat can efficiently be removed by using a solvent with a large heat capacity. Further, since reaction is executed at a low temperature, there is an advantage that methanol which is a product is formed as a liquid and therefore is not restricted with respect to equilibrium, and a perfect reaction without requiring a recycling process of unconverted gas can be expected. Thus, a low-temperature liquid-phase synthesis process is deemed promising as a low-cost production technology of methanol. Regarding liquid-phase methanol synthesis, since an equilibrium is decided by the vapor pressure of methanol and by unconverted carbon monoxide and hydrogen, reaction progresses when carbon monoxide and hydrogen are higher in pressure than carbon monoxide and hydrogen which are in equilibrium with methanol vapor in vapor-pressure equilibrium exist.

As for liquid-phase synthesis of methanol, a conventional method for suspending a copper zinc oxide catalyst in a liquid phase (S. Lee, “Methanol Synthesis Technology”, (1990) CRC press.; L. Fan, Y. Sakaiya, and K. Fujimoto, Appl. Catal. A, 180, L11 (1999); N. Tsubaki, M. Ito, and K. Fujimoto, J. Catal., 197, 224 (2001); N. Tsubaki, et al., Catal. Comm., 2, 213 (2001); D. Dombek, J. Organomet. Chem., 372, 151 (1989); T. Deguchi, Y. Kiso, T. Onoda, and Y. Watanabe, “Progress in Cl Chemistry in Japan”, Kodansha-Elsevier (1989), p. 67), a method of using an Ni catalyst or Cu-based catalyst along with alkali metal alcoxide (low-temperature liquid-phase methanol synthesis), and the like have been reported. The low-temperature liquid-phase methanol synthesis using alkali metal alcoxide has been reported as a process for obtaining two methanol molecules from one methanol molecule, by carbonylating methanol into methyl formate and then hydrocracking the methyl formate (Formulae 1-2 and 1-3). Regarding the low-temperature liquid-phase methanol synthesis through methyl formate, the U.S. patent application (J. A. Christiansen, U.S. Pat. No. 1,302,011 (1919)) by Christiansen in 1919 is deemed to be the oldest, and the process has been actively studied since the late 1970s. As catalysts for low-temperature liquid-phase methanol synthesis, BNL catalysts (NaH—ROH—Ni(CH₃COO)₂ and NaH—RONa—Ni(CH₃COO)₂), alkali metal alcoxide-tetracarbonyl nickel catalysts (R. Sapienza et al., U.S. Pat. No. 4,614,749 (1986); R. Sapienza et al., U.S. Pat. No. 4,619,946 (1986); R. Sapienza et al., U.S. Pat. No. 4,623,634 (1986); D. Mahajan, R. A. Sapienza, W. A. Slengeir, and T. E. O'Hare, U.S. Pat. No. 4,935,395 (1990); M. Marchionna, L. Basini, A. Aragno, M. Lami, and F. Ancillotti, J. Mol. Catal., 75, 147 (1992); S. Ohyama, Preprints, Div. Pet. Chem., ACS, 38, 100 (1993); Seiichi Ohyama, Central Research Institute of Electric Power Industry report T 89034 (1990); T 90027 (1991); T 91086 (1992); T 94039 (1995); S. Ohyama, Appl. Catal. A, 181, 87 (1999)), NiCl₂-t-C₄H₉₀Na catalyst (Mitsui Petrochem., Japan Patent, 81/169,934 (1981); S. T. Sie, E. Dreant, and W. W. Jager, BR Patent, 88/14896 (1989); Eur. Pat., 285,228 (1988)), or the like have been reported.

Formula 2

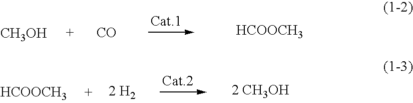

With respect to the catalysts reported, the reaction temperature is 373-473 K, the pressure is 5 MPa, and the methanol space time yield per catalytic layer volume is 0.1-0.9 kgL⁻¹h⁻¹. Among those, BNL catalysts developed by Brookhaven National Laboratory in the USA are catalysts that are highly active on such a low-temperature condition as 373 K, at which methanol synthesis is thermodynamically advantageous, and that show such excellent catalytic characteristics of 90% or more carbon monoxide conversion and 99% methanol selectivity, and also high space time yield (STY). The catalysts developed by BNL are classified into three kinds that are a nickel base, a palladium base and a cobalt base, and are composed of metal acetate, sodium hydride and alcohol, respectively; and tetrahydrofuran, triethylene glycol dimethyl ether (triglyme) and the like are used as solvents. As a result of a double-check by Ohyama et al., only the nickel-based catalyst shows high activity from among the three kinds of catalysts, and a carbon monoxide conversion of 88% and a methanol selectivity of 99% have been obtained in an hour reaction at 373 K.

Wender et al. have synthesized methanol from synthetic gas by combining alkali metal alcoxide with a Cu—Cr—Mn based liquid-phase heterogeneous catalyst, with methanol serving as a solvent, on condition of 393-453 K in temperature and 3.5-6.5 MPa in pressure. The fact that, in the co-presence of a copper chromite based catalyst, various alkali metal salts can be used instead of alkali metal alcoxide has been shown, however, a result that they are low-active in comparison with a BNL process has been reported.

As described thus far, low-temperature liquid-phase methanol synthesis using a nickel-sodium methoxide composite catalytic system progresses through methyl formate, which is a mechanism having two steps. Methanol carbonylation reaction in the first step progresses with sodium methoxide dissolved in methanol serving as a catalyst (Formula 2-1). This reaction is a basic reaction in a methyl formate method for formic acid production process and was published by BASF Ltd. in 1925. This reaction is an exothermic reaction in which, industrially speaking, synthesis takes place with sodium methoxide of an approximately 2 wt % concentration serving as a catalyst in a liquid phase on condition of 353 K and 4 MPa of carbon monoxide, attaining a carbon monoxide conversion of 95%, a methanol conversion of 30%, a methyl formate selectivity of 99% and a space time yield of 800 gL⁻¹h⁻¹. Studies have intensively made on methyl formate synthesis and most of them relate to alkali metal methoxide; and further there have been studies using anion-exchange resin (D. L. Daremsbourg, U.S. Pat. No. 4,100,360, 1978; M. D. Giroramo, et al., Catal. Lett., 38, 127 (1996)), using an organic strong base, using a transition metal carbonyl complex, and so forth. Girolamo et al. have executed a methanol carbonylation reaction using the strongly-basic anion-exchange resin Amberlyst A26 on condition of 343 K and 5 MPa of carbon monoxide, and have obtained such high catalytic activity that the carbon monoxide conversion is 83% and the TOF 73 h⁻¹.

Formula 3
CH₃OH+CO→HCOOCH₃ [symbol] H⁰=−29.1 kJmol⁻¹ (2-1)

In addition, the inventors have reported several results of studies in relation to the present invention (Hidenori Kobayashi, Ken-ichi Aika, The 31st petroleum and petrochemistry debate, Drafts, B32, p. 61-62 (2001). Hidenori Kobayashi, Daisuke Hiramoto, Ken-ichi Aika, The 90th catalyst debate, 1P03, Debate A drafts p 3 (2002) Sept. 18-21. Ken-ichi Aika, Daisuke Hiramoto, The 32th petroleum and petrochemistry debate, C43, p 223 (2002). Ken-ichi Aika, Lee Eun-sook, Makoto Nishikubo, Ken Kiyono, Hidenori Kobayashi, Daisuke Hiramoto, Koji Inazu, The 91th catalyst debate B, 2A04(TB2), Lecture drafts p 126-128, Yokohama National University Mar. 26-27 (2003).).

### DISCLOSURE OF INVENTION

As described above, liquid-phase methanol synthesis is a promising reaction method for the future, and a lot of potential catalysts therefor have been reported. However, all the catalysts include soluble catalytic ingredients. For example, in low-temperature liquid-phase methanol synthesis using a nickel-sodium methoxide composite catalytic system, sodium methoxide not only serves as a catalyst in a methanol carbonylation reaction, which is the first step in the reaction, but also has an important function in a methyl formate hydrogen decomposition reaction, which is the second step. However, since sodium methoxide is a homogeneous catalyst which dissolves in methanol, a separation process between a catalyst and a product is required. If a catalyst is 100 percent solidified, with gas of carbon monoxide and hydrogen being pushed into a semibatch reactor or a flow reactor, methanol by almost 100 percent is created, which is a chemical reaction process without any separation process where a product can be made only by discharging the liquid from a container bottom (semibatch reactor) or a reaction tube exit (flow reactor). In addition, since recirculation is unnecessary, nitrogen gas may be contained, allowing air to be used instead of pure oxygen in partial oxidation in a synthetic gas production process.

However, such a solid catalyst has not yet been developed.

The present invention is made in view of the above problems, and aims at providing a novel solid catalyst. Further, the present invention aims at providing a novel reactor. Further, the present invention aims at providing a novel method of producing methanol.

As a result of intensive studies in an attempt to resolve the above-mentioned problems, the inventors of the present invention et al. have found out that methanol is produced by using a heterogeneous catalyst in which an ion-exchange resin anion-exchanged to be an alcoxide type has been combined with a transition metal, hence the present invention is made.

Specifically, the gist of the present invention is a solid catalyst in which an ion-exchange resin anion-exchanged to be an alcoxide type has been combined with a transition metal.

Hereupon, it is preferable that as a transition metal/metals, copper or several kinds of metals including copper are provided for combination. Further, it is preferable that, as an ion-exchange resin, ones whose allowable temperature range is from 333 to 453 K, are provided for combination. It is further preferable that the allowable temperature range be from 353 to 423 K. Further, it is preferable that such a combination having the mass ratio of an ion-exchange resin to a transition metal catalyst being in the range between 1/100 and 100/1. Further, it is more preferable that such a combination having the mass ratio of an ion-exchange resin to a transition metal catalyst being in the range between 1/10 and 10/1. Further, it is preferable to have a solid catalyst working in a solution of methanol and a polar organic solvent mixed together. Further, it is preferable to combine an ion-exchange resin having a methoxide anion as an alcoxide to be exchanged.

Furthermore, the gist of the present invention is a reactor containing a solid catalyst in which an ion-exchange resin anion-exchanged to be an alcoxide type is combined with a transition metal.

Furthermore, the gist of the present invention is a method of producing methanol, in which carbon monoxide and hydrogen are made to react together in the presence of a solid catalyst where an ion-exchange resin anion-exchanged to be an alcoxide type is combined with a transition metal.

The present invention provides such effectiveness as follows.

The present invention can provide a novel solid catalyst by combining an ion-exchange resin anion-exchanged to be an alcoxide type with a transition metal.

The present invention can provide a novel reactor by making the reactor contain a solid catalyst in which an ion-exchange resin anion-exchanged to be an alcoxide type is combined with a transition metal.

The present invention can provide a novel method of producing methanol by making carbon monoxide and hydrogen react together in the presence of a solid catalyst in which an ion-exchange resin anion-exchanged to be an alcoxide type is combined with a transition metal.

### BRIEF DESCRIPTION OF DRAWINGS

FIG. 1 is a diagram showing temporal changes of pressure and temperature in low-temperature methanol synthesis at 393 K by means of a Raney copper-TSA1200 composite catalyst. (O) pressure change; (▪) temperature change; reaction condition: 393 K, 7.5 h, 5.0 Mpa (H₂/CO/Ar=8/4/1); a total of 100 ml is made by adding triglyme to 2 g of Raney copper, 20 ml of TSA1200 and 10 ml of methanol.

FIG. 2 is a diagram showing temporal changes of pressure and temperature in low-temperature methanol synthesis at 423 K by means of a Raney copper-TSA1200 composite catalyst. Temporal changes of temperature (▪) and pressure (O) are shown when methanol synthesis has been executed at 423 K (150° C.) by means of 5.0 MPa of synthetic gas (H₂/CO/Ar=8/4/1) in 100 ml of solution made by adding triglyme to Raney copper (2.0 g), TSA1200 (20 ml) and methanol (10 ml). After a four-hour reaction, the synthetic gas which has diminished is added to be 5.0 MPa once again, and yet another four-hour reaction is executed.

FIG. 3 is a diagram showing temporal changes of pressure and temperature in low-temperature methanol synthesis at 393 K by means of a Raney copper-TSA1200 composite catalyst. Temporal changes of temperature (▪) and pressure (O) are shown when methanol synthesis has been executed at 393 K (120° C.) by means of 5.0 MPa of synthetic gas (H₂/CO/Ar=8/4/1) in 100 ml of solution made by adding triglyme to Raney copper (2.0 g), TSA1200 (20 ml) and methanol (10 ml). After a four-hour reaction, the synthetic gas which has diminished is added to be 5.0 MPa once again, and yet another four-hour reaction is executed.

FIG. 4 is a diagram showing temporal changes of pressure and temperature in low-temperature methanol synthesis at 373 K by means of a Raney copper-TSA1200 composite catalyst. Temporal changes of temperature (▪) and pressure (O) are shown when methanol synthesis has been executed at 373 K (100° C.) by means of 5.0 MPa of synthetic gas (H₂/CO/Ar=8/4/1) in 100 ml of solution made by adding triglyme to Raney copper (2.0 g), TSA1200 (20 ml) and methanol (10 ml). After a four-hour reaction, the synthetic gas which has diminished is added to be 5.0 MPa once again, and yet another four-hour reaction is executed.

FIG. 5 is a diagram showing temporal changes of pressure and temperature in low-temperature methanol synthesis at 373 K by means of a Raney copper-TSA1200 composite catalyst. Temporal changes of temperature (▪) and pressure (O) are shown when methanol synthesis has been executed at 373 K (100° C.) by means of 5.0 MPa of synthetic gas (H₂/CO/Ar=8/4/1) in 100 ml of solution made by adding triglyme to Raney copper (2.0 g), TSA1200 (20 ml) and methanol (10 ml). After a four-hour reaction, the synthetic gas which has diminished is added to be 5.0 MPa once again, and yet another four-hour reaction is executed; subsequently, the synthetic gas is further added by 5.0 MPa, and a 10-hour reaction is executed additionally.

FIG. 6 is a diagram showing Raney copper amount dependency in low-temperature methanol synthesis at 393 K by means of a Raney copper-TSA1200 composite catalyst ((A) 1 g, (O) 2 g, (O) 4 g). Reaction condition: 393 K, 4 h, 5.0 Mpa (H₂/CO/Ar=8/4/1); a liquid amount of 100 ml is made by adding 20 ml of TSA1200, 10 ml of methanol, and triglyme.

FIG. 7 is a diagram showing pressure changes and temperature changes in methanol synthesis (a) and in methyl formate hydrogenation (b) by means of a Raney copper-sodium methoxide composite (soluble) catalyst ((O) pressure, (▪) temperature). Reaction condition: 393 K, 2 h, Raney copper 2 g, NaOCH₃ 2.2 g (41 mmol) (a) 5.0 Mpa (H₂/CO/Ar=8/4/1), methanol 30 ml, triglyme 70 ml (b) 3.5 Mpa (H₂/Ar=8/1), methyl formate 7.7 ml, methanol 22.3 ml, triglyme 70 ml.

### BEST MODE OF CARRYING OUT THE INVENTION

Hereinafter, an embodiment of the present invention will be explained.

A solid catalyst of the present invention is the catalyst in which an ion-exchange resin anion-exchanged to be an alcoxide type is combined with a transition metal.

It is preferable that the allowable temperature range of the ion-exchange resin be between 333 and 453 K, and more preferable that the foregoing be in the range of 353 to 423 K. If the allowable temperature is 333 K or above, the reaction temperature can be set high, allowing the reaction rate to be high, so that there are such advantages of only a small amount of catalyst needed, heat generated by the reaction, which can be utilized more efficiently, and so forth. If the allowable temperature is 453 K or below, reaction must take place at 453 K or below, but there are such advantages that operation can be performed on a reaction condition where the equilibrium conversion is high, a wider selection of heat-resisting resins is possible, and so forth. If the allowable temperature range is from 353 to 423 K, the above-mentioned effectiveness becomes conspicuous.

It should be noted that the allowable temperature of an ion-exchange resin means the highest temperature at which methanol synthesis is executed, using Raney copper and an ion-exchange resin on the following condition, and the amount of hydrogen consumed is 10 mmol or more or the amount of methanol formed is 5 mmol or more in four hours.

Methanol synthesis for measuring an allowable temperature is executed in such a manner as follows. 100 ml of solution made by adding triglyme to Raney copper (2.0 g), an ion-exchange resin (20 ml) and methanol (10 ml) is poured into a batch-type magnetic induction agitation autoclave (234 ml in inner volume) made of stainless steel (SUS 316); reaction is executed by means of 5.0 MPa of synthetic gas (H₂/CO/Ar=8/4/1) at a predetermined temperature for four hours; and changes of temperature and pressure in the reaction are closely watched and gas components and liquid components are analyzed after the reaction.

Any ion-exchange resin which can be anion-exchanged to an alcoxide type may be employed. For example, there are polystyrene resin, polyvinyl resin and like to which amines such as a quaternary amine (—CH₂R₁R₂R₃N⁺X⁻) and a tertiary amine (—CH₂R₁R₂NH⁺X⁻) have been bonded.

Among them, ion-exchange resins with the following structure are preferable.
—C₆H₄(CH₂)ₙN(CH₃)₃⁺X⁻

where n represents an integer of 1 or more and 10 or less, and X⁻ represents an anion. In the formula, n stands at a minimum of 1 or above, preferably 3 or above, and normally stands at a maximum of 10 or below, preferably 8 or below, more preferably 6 or below, most preferably 5 or below. Specifically speaking, it is preferable to have such chemical structures as —C₆H₄—(CH₂)₃N(CH₃)₃⁺Cl⁻, —C₆H₄—(CH₂)₄N(CH₃)₃⁺Cl⁻ and —C₆H₄—(CH₂)₅N(CH₃)₃⁺Cl⁻. There is no particular limitation upon particle diameters of an ion-exchange resin; however, the average particle diameter is normally from 100 to 2000 μm.

As an alcoxide with which exchange takes place, a methoxide anion is preferable, but is not particularly limited thereto. As other alcoxides, there are alcoholic alcoxides of 1 to 20 in carbon number such as ethanol, n-propanol, i-propanol, n-butyl alcohol, sec-butyl alcohol, tert-butyl alcohol, i-pentyl alcohol, tert-pentyl alcohol, n-octyl alcohol, lauryl alcohol, cetyl alcohol, cyclopentanol, cyclohexanol, allyl alcohol and benzyl alcohol; for example, it is possible to employ polyalcoholic alcoxides having two to eight hydroxyl groups of two to twenty in carbon number such as ethylene glycol, propylene glycol, diethylene glycol, 1,3-propanediol, 1,3-butanediol, 1,4-butanediol, 1,6-hexanediol, cyclohexanediol, trimethylolpropane, glycerin and pentaerythritol, and the like.

As for transition metals, copper or several kinds combined of metals including copper are preferable. There is a case where a transition metal having the optimum combination differs according to the chemical constitution or particle diameter of ion-exchange resin. As transition metals, copper, zinc, zirconium, chromium, gold, silver, nickel, cobalt, iron, ruthenium, palladium, rhodium, platinum and the like can be employed. Further, there is a case where as a supporting role, an oxide of a typical metal such as aluminum, magnesium, calcium, potassium or sodium or an oxide of a rare-earth metal such as lanthanum or cerium could be effective. There is no particular limitation upon the amount of oxide of a typical metal added; however, it is preferable to be a fifth of the mass of a transition metal or less. There is no particular limitation upon a method to add, and a typical method such as an impregnation method or precipitation method is used.

It is preferable that the form of a transition metal be a Raney type, a powder type or a supported type, and that a carrier be alumina, silica, activated charcoal, an ion-exchange resin or the like. There is no particular limitation upon the size, however, 50 μm or more is preferable on the grounds that separation from a solvent becomes easy.

It is preferable that the mass ratio of an ion-exchange resin to a transition metal catalyst be between 1/100 and 100/1. If the mass ratio is 1/100 or more, there is such an advantage that the rate of an intermediate methyl formate formation reaction by means of the carbonylation of methanol, which is a reaction in the first step, becomes higher. If the mass ratio is 100/1 or less, there is such an advantage that the rate of a hydrogenation reaction of methyl formate, which is a reaction in the second step, becomes higher.

It is more preferable that the mass ratio of an ion-exchange resin to a transition metal catalyst be between 1/10 and 10/1. If the mass ratio is 1/10 or more, there is such an advantage that the rate of a carbonylation reaction of methanol becomes high enough, causing a favorable balance between that rate and the hydrogenation rate of methyl formate formed. If the mass ratio is 10/1 or less, there is such an advantage that the hydrogenation reaction rate of methyl formate becomes high enough, causing a favorable balance between that rate and the carbonylation reaction rate of methanol.

Reaction can be executed with a standard reactor used for gas-liquid-solid catalytic reactions; and a batch/semibatch reaction method using a tank-like reactor (autoclave), or a continuous reaction method using a tubular reactor can be used. A product can be obtained by introducing gas of carbon monoxide and hydrogen into a reactor, and by discharging a methanol-containing liquid which has been formed from a container bottom (tank-like reactor) or from a reaction tube exit (tubular reactor).

As regards carbon monoxide and hydrogen that are raw materials, a synthetic gas can be used, and it is preferable that the volume ratio of carbon monoxide to hydrogen be 1:1 to 1:3. Further, inactive gasses such as nitrogen gas may be included. Thus, as a raw material in partial oxidation in a synthetic gas production process, air that costs less can be used, as compared to pure oxygen in related art.

It is preferable that a solvent used for reaction, which is not particularly limited, include methanol or dimethyl ether. Further, other organic solvents than those can be mixed for reaction. As organic solvents, polar solvents are preferable, more preferably methyl formate and triglyme can be used.

It is preferable that the reaction temperature, which is not particularly limited, be in the range of 333 to 453 K, more preferably in the range of 353 to 423 K. With respect to the reaction pressure, the optimum range varies depending on the amount of catalyst and the reaction temperature; if the pressure is too low, the reaction rate will be low, and so the reaction pressure is preferably 0.1 MPa or more, more preferably 1 MPa or more, and is normally 50 MPa or less, preferably 10 MPa or less.

Although not particularly limited, the volume ratio of a liquid phase to a gas phase in a reactor is normally 0.1 or more and 10 or less.

Since a catalyst does not dissolve in methanol formed or solvent, separation is easily made possible by installing a filter inside or outside a reactor. A methanol-containing solution from which a catalyst is separated can be separated from a side product and solvent by a typical refining operation such as distillation.

Further, since highly pure methanol can be obtained only by separating a catalyst from a reaction solution provided that methanol is used as a solvent and further improvement in the selectivity to methanol in a reaction has been achieved, it is possible that the methanol will be able to be used for other purposes without particularly being refined.

Thus, according to this embodiment, liquid-phase methanol synthesis is made possible on a mild condition, by making a metal, particularly Raney copper, mixed with an ion-exchange resin of an alcoxide type.

The above is invaluable as a forerunner of a technology with which liquid-phase methanol can be synthesized continuously in one step without separating reactants.

It should be noted that the present invention is not limited to the above-mentioned embodiment, and needless to say can employ various other structures without deviating from the gist of the present invention.

Next, practice examples according to the present invention will be explained specifically. Note that, needless to say, the present invention is not limited to these practice examples.

### PRACTICE EXAMPLE 1

### Ion Exchange

Ion-exchange resin reagents used: Amberlyst A26 Rohm & Haas Co. and DIAION TSA1200 (Lot 0 K681) Mitsubishi Chemical Corporation.

A strongly-basic anion-exchange resin was ion-exchanged to be CH₃O^{-. 150} ml of Cl⁻-type anion-exchange resin hydrated was measured and taken with a measuring cylinder, and was moved to a column made of Pyrex, using distilled water. First, approximately 1 molL⁻¹ of HCl solution was passed by the amount of 10 times the volume of the resin and by 2 mlmin.⁻¹ (reverse generation). After that, distilled water was passed by the amount 10 times and by 10 mlmin.⁻¹ to be rinsed. Next, approximately 1 molL⁻¹ of NaOH solution was passed by the amount of 20 times the volume of the resin and by 1 mlmin.⁻¹ (generation). After that, distilled water was passed by the amount of 20 times and by 10 mlmin.⁻¹ to be rinsed. Subsequently, approximately 1 molL⁻¹ of NaOCH₃ methanol solution was passed by the amount of 20 times the volume of the resin and by 1 mlmin.⁻¹ (activation). After that, methanol was passed by the amount of 20 times and by 10 mlmin.⁻¹ to be rinsed.

SEM-EDX measurement was carried out upon ion-exchange resins of each ion type. Gold was deposited on anion-exchange resins of each ion type on the same condition and the measurement was carried out. An estimation of the Cl⁻ residual ratio of an ion-exchange resin was made from the peak area ratio of gold to chlorine in a spectrum obtained, determining the replacement ratio from the Cl⁻ type to the OH⁻ type. Regarding the replacement ratio from the Cl⁻ type to the OH⁻ type, Amberlyst A26 turned out to be 84.1%, while DIAION TSA1200 turned out to be 100%.

### PRACTICE EXAMPLE 2

### Raney Copper Catalyst-Anion-exchange Resin Catalytic System

As regards Raney copper, Raney copper already alloy developed and made into a slurry form (pH>9), as the commercially available product (Aldrich Co.), was used. After distilled water and THF had given to clean several times, respectively, THF was distilled away using a rotary evaporator, and the copper was mixed with a reaction solution under a nitrogen current.

An ion-exchange resin, which is a catalyst, was measured and taken with a measuring cylinder by a predetermined amount (typically 20 ml), and a reaction solution was made by adding 10 ml of methanol. Subsequently, a total of 100 ml was made by adding triglyme, which is a solvent. A metal catalyst was used, being mixed with the solution in an autoclave. If sodium methoxide and metal were exclusively used as a catalyst, a total of 100 ml was made by adding triglyme to 30 ml of methanol, and the catalyst was mixed with a solution in an autoclave to be used. Reaction gas was made to be H₂/CO/Ar=8/4/1, and was introduced by 5 MPa. Ar was used as an internal standard. The agitation rate was made 800 rpm.

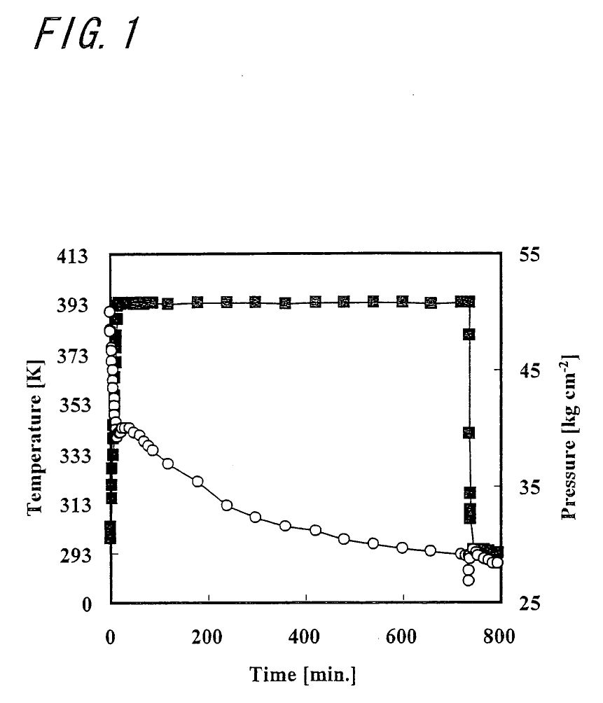

The result of low-temperature liquid-phase methanol synthesis using a Raney copper-TSA1200 composite catalytic system is shown in Table 1. Further, temporal changes of pressure and temperature in the reaction are shown in FIG. 1. In approximately 15 minutes after the start of the reaction, a sharp pressure decrease was seen, and afterward the pressure gradually decreased. 58.4 mmol of methanol was formed at 393 K in a 7.5-hour reaction, and the carbon monoxide conversion was 67.2% and the methanol selectivity 78.1%. Liquid-phase methanol was successfully synthesized from synthetic gas, using a solid (composite) catalyst, at 393 K.

TABLE 1
Table 1 Low-temperature Methanol Synthesis At 393 K By
Means Of Raney Copper - TSA1200 Composite Catalyst

| | | Methanol | Product [mmol] | | |
|---|---|---|---|---|---|
| | CO Conversion | Selectivity | | Methyl | |
| Time [h] | [%] | [%] | Methanol | Formate | Methane |
| 4 | 72.3 | 70.4 | 32.9 | 6.9 | — |
| 7.5 | 67.2 | 78.1 | 58.4 | 8.2 | — |

Reaction condition: 393 K, 5.0 Mpa (H₂/CO/Ar=8/4/1); a total of 100 ml is made by adding triglyme to 2 g of Raney copper, 20 ml of TSA1200 and 10 ml of methanol.

### PRACTICE EXAMPLE 3

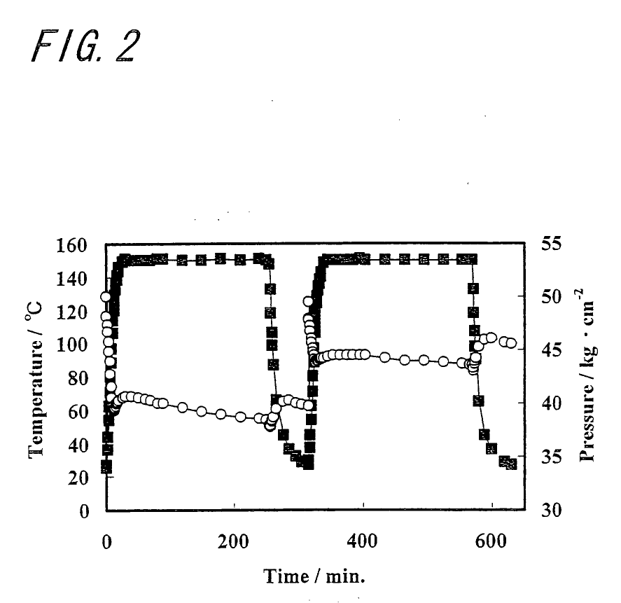

FIG. 2 shows an example in which the catalyst produced in Practice Examples 1 and 2 is repetitively used at 423 K (150° C.) in reaction temperature, and reaction was executed on the same condition as in Practice Example 2 except that the reaction time is different.

Regarding both the first and second times, the first sharp pressure decrease is mainly due to decrease of CO and formation of methyl formate caused by the carbonylation of methanol, and the subsequent gradual pressure decrease is mainly due to decrease of H₂ and formation of MeOH caused by the hydrogenation of methyl formate.

Regarding the first time, in four hours, the amount by which CO had decreased was 29 mmol (46% in conversion), the amount by which methyl formate had been formed was 21 mmol, the amount by which MeOH had been formed was 41 mmol, and the amount by which hydrogen had decreased was 47 mmol; regarding the second time, the amount by which CO had decreased was 8 mmol (22% in conversion), the amount by which methyl formate had been formed was 7 mmol, the amount by which MeOH had been formed was 20 mmol, and the amount by which hydrogen had decreased was 20 mmol.

As a result, the allowable temperature of TSA1200 in accordance with the above-described definition is found out to be higher than 423 K.

### PRACTICE EXAMPLE 4

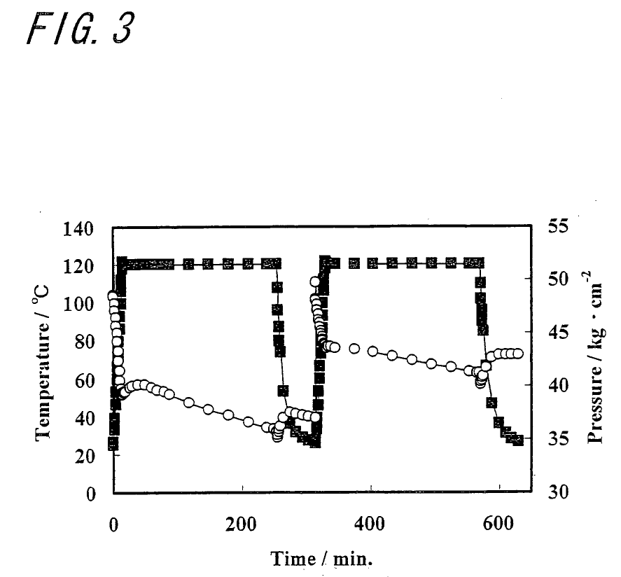

Reaction was executed on the same condition as Practice Example 3 except 393 K (120° C.). The result thereof is shown in FIG. 3. Here, the amount by which CO had decreased regarding the first time was 42 mmol (61% in conversion), and the amount by which CO had decreased regarding the second time was 21 mmol (34% in conversion). At the time when ending the second time, the amount by which methyl formate had been formed was 37 mmol, the amount by which MeOH had been formed was 45 mmol, and the amount by which hydrogen had decreased was 62 mmol.

### PRACTICE EXAMPLE 5

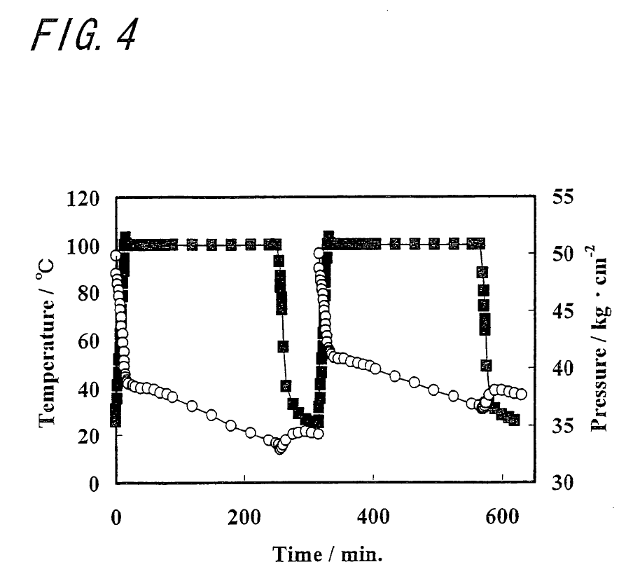

Reaction was executed on the same condition as Practice Example 2, except that the reaction temperature, the reaction time and the number of reactions are different. FIG. 4 shows the result of an experiment in which the reaction temperature was 373 K, the reaction time was set as the diagram shows, and the reaction was executed twice in a consecutive manner. Here, the amount by which CO had decreased regarding the first time was 54 mmol (76% in conversion), and the amount by which CO had decreased regarding the second time was 40 mmol (61% in conversion). The amount by which methyl formate had been formed was 64 mmol, the amount by which MeOH had been formed was 34 mmol, and the amount by which hydrogen had decreased was 74 mmol. It has been found out that low-temperature liquid-phase methanol synthesis progresses for a long time at 373 K as well.

### PRACTICE EXAMPLE 6

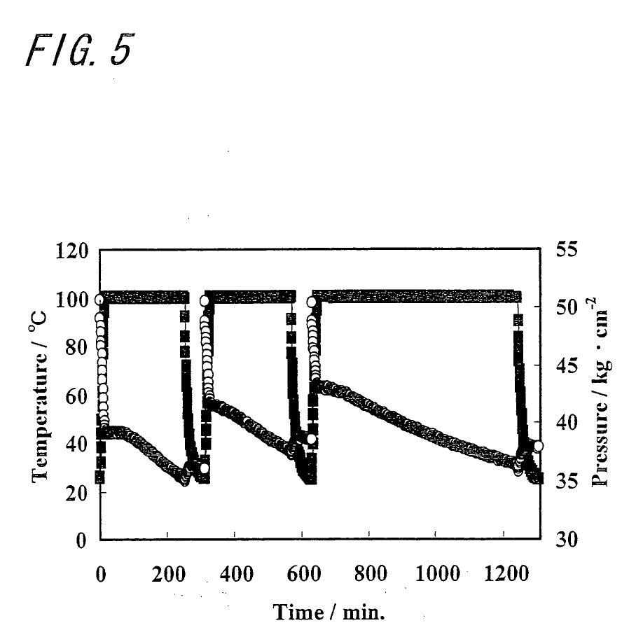

Except for the number of reactions, the same reaction as Practice Example 5 was executed; here, the same catalyst was used to execute reactions three times consecutively, and further, the third reaction was executed for 10 hours. The result thereof is shown in FIG. 5. Reaction progresses similarly regarding the third time as well, and it is indicated that the synthesis at 373 K progresses for a longer time.

### PRACTICE EXAMPLE 7

### Anion-Exchange Resin Dependency

In order to examine the ion-exchange resin amount dependency of catalytic activity in low-temperature liquid-phase methanol synthesis using a Raney copper-TSA1200 composite catalytic system, reaction was executed with the amount of ion-exchange resin being changed. The result thereof is shown in Table 2. If there is no ion-exchange resin and there is only Raney copper, methanol is not generated. The methanol formation amount and the methanol selectivity increased synchronously with increase in the ion-exchange resin amount. It has been found out that TSA1200 is a necessary constituent.

TABLE 2
Low-temperature Methanol Synthesis At 393 K
By Means Of Raney Copper - TSA1200 Composite
Catalyst: Resin Amount (TSA1200) Dependency

| Amount | | Methanol | Product [mmol] | | |
|---|---|---|---|---|---|
| Of Resin | CO Conversion | Selectivity | | Methyl | |
| [ml] | [%] | [%] | Methanol | Formate | Methane |
| 0 | ˜0 | ˜0 | ˜0 | ˜0 | ˜0 |
| 10 | 73.1 | 17.3 | 5.7 | 13.7 | ˜0 |
| 20 | 72.3 | 70.4 | 32.9 | 6.9 | ˜0 |
| 40* | 78.0 | 72.2 | 54.6 | 10.5 | ˜0 |

Reaction condition: 393 K, 4 h, 5.0 Mpa (H₂/CO/Ar=8/4/1); a total of 100 ml is made by adding triglyme to 2 g of Raney copper and 10 ml of methanol.

### PRACTICE EXAMPLE 8

### Raney Copper Amount Dependency

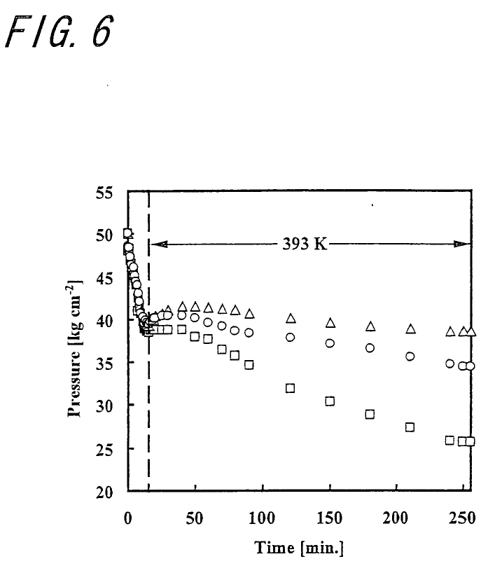

In order to examine the Raney copper amount dependency of catalytic activity in low-temperature liquid-phase methanol synthesis using a Raney copper-TSA1200 composite catalytic system, reaction was executed with the amount of Raney copper being changed. The result thereof is shown in FIG. 3. As the Raney copper amount increased, the methanol formation amount and the methanol selectivity increased. It has been proved that Raney copper is a requisite constituent. On the other hand, FIG. 6 shows temporal pressure changes in reaction. In Table 3, the carbon monoxide conversion is almost constant; regarding the temporal pressure changes in FIG. 6, however, a large difference is observed in pressure decrease behavior approximately 15 minutes after the start of the reaction, and the pressure decrease rate becomes greater as the Raney copper amount increases. This means that in the first 15 minutes the carbonylation of methanol by TSA1200 progresses mainly and that from 15 minutes onward the hydrogenation of methyl formate progresses mainly, and so hydrogen consumption takes place. It has been assumed that this is because the effects of Raney copper start to exist in this latter stage.

TABLE 3
Low-temperature Methanol Synthesis At 393 K
By Means Of Raney Copper - TSA1200 Composite
Catalyst: Raney Copper Amount Dependency

| Amount | | Methanol | Product [mmol] | | |
|---|---|---|---|---|---|
| Of Copper | CO Conversion | Selectivity | | methyl | |
| [g] | [%] | [%] | Methanol | formate | Methane |
| 1 | 69.5 | — | — | 20.7 | trace |
| 2 | 72.3 | 70.4 | 32.9 | 6.9 | — |
| 4 | 73.4 | 72.2 | 44.9 | 8.6 | — |

Reaction condition: 393 K, 4 h, 5.0 Mpa (H₂/CO/Ar=8/4/1); a total of 100 ml is made by adding triglyme to 20 ml of TSA1200 and 10 ml of methanol.

### PRACTICE EXAMPLE 9

Methanol synthesis was executed on the same condition as Practice Example 2, except that Amberlyst A26 was used instead of TSA1200 and the reaction was made four hours. The CO consumption amount was 41 mmol, the methyl formate formation amount was 26 mmol, and the methanol formation amount was 29 mmol. Further, the hydrogen consumption amount was 28 mmol. It has been found out that although Amberlyst A26 does not generate methanol if combined with nickel (Reference Example 3), Amberlyst A26 can synthesize methanol on the same condition at 393 K if combined with copper.

Further, as a result, it has been found out that the allowable temperature of Amberlyst A26 in accordance with the above-described definition is higher than 393 K.

### REFERENCE EXAMPLE 1

### Methyl Formate Hydrogenation Decomposition Reaction Using Raney Copper

Table 4 shows the result of a methyl formate hydrogenation decomposition reaction with Raney copper used as a catalyst. Only with Raney copper, methyl formate hydrogenation decomposition reaction progressed; at 423 K and 393 K, the methanol formation amount was 106.1 mmol and 39.3 mmol, respectively, and the methyl formate conversion was 67.5% and 32.8%, respectively. This has proved that the latter reaction step (described previously) is caused by Raney copper.

TABLE 4
Methyl Formate Hydrogenation Decomposition
Reaction By Means Of Raney Copper

| | | Methyl | | | | |
|---|---|---|---|---|---|---|
| Temper- | | Formate | Methanol | Formation | | |
| ature | Time | Conversion | Selectivity | Amount [mmol] | | |
| [K] | [h] | [%] | [%] | Methanol | CO | Methane |
| 423 | 3 | 67.5 | 99.9 | 106.1 | ˜0 | ˜0 |
| 393 | 5 | 32.8 | 99.9 | 39.3 | ˜0 | ˜0 |

Reaction condition: 5.0 Mpa (H₂/Ar=8/1), Raney copper 2 g, methyl formate 7.7 ml, triglyme 92.3 ml.

### REFERENCE EXAMPLE 2

Methanol carbonylation reaction, with solvent added, was executed. It was assumed that low-temperature liquid-phase methanol synthesis could have a solvent effect; therefore, in order to examine the effects of an additive solvent on carbonylation that is a prior reaction, methanol carbonylation reaction using a solvent (triglyme) was executed, and influences by a solvent of an ion-exchange resin were examined. The result thereof is shown in FIG. 5. Catalytic activity, which had decreased by using a solvent, increased by increasing the amount of ion-exchange resin. It has been confirmed that with a sufficient amount of ion-exchange resin, activity can be maintained.

TABLE 5
Solvent Effect On Methanol Carbonylation
Reaction By Amberlyst A26

| | | | | Methyl |
|---|---|---|---|---|
| | | | CO | Formate |
| Amount Of | Solvent | CO | Consumption | Formation |
| Resin | Triglyme | Conversion | Amount | Amount |
| [ml] | [ml] | [%] | [mmol] | [mmol] |
| 5 | — | 69.6 | 166.6 | 216.2 |
| 5 | 70 | 24.1 | 64.1 | 46.3 |
| 10 | 70 | 58.3 | 142.8 | 131.7 |

Reaction condition: 373 K, 2 h, 5.0 Mpa (CO/Ar=4/1, CO c.a. 240 mmol).

### REFERENCE EXAMPLE 3

### Low-Temperature Liquid-Phase Methanol Synthesis Using Nickel-Anion-Exchange Resin Composite Catalytic System

As a prior treatment for a nickel fine powder catalyst, hydrogen reduction was executed. A catalyst was measured and introduced into a reaction tube; using a closed circulation system reactor made of Pyrex, after the catalytic sample underwent at 473 K the evacuation of gas to a vacuum, 0.07 MPa of hydrogen was introduced and was circulated at 473 K for one hour through a cold trap of liquid nitrogen for trapping water and the like which were thought to be generated in the reduction on the oxidized surface of the catalyst. After that, while the evacuation of gas to a vacuum was continued, the temperature was cooled down to room temperature; and, under a nitrogen current, the reaction tube was removed to execute mixture with a reaction solution.

As regards Raney nickel, an alloy of Al(50 wt %)-Ni(50 wt %) was developed in 5N of sodium hydroxide solution at 323 K for 30 minutes, and later at 353 K for 17 hours. In this process, aluminum is eluted, and hydrogen is generated.

After the development, the Raney nickel was cleaned by water until the pH became 7; next, after being cleaned by tetrahydrofuran (THF), THF was distilled away using a rotary evaporator, and the Raney nickel was mixed with a reaction solution under a nitrogen current. The result of a low-temperature liquid-phase methanol synthesis reaction using nickel fine powder and a Raney nickel-anion-exchange resin composite catalytic system is shown in Table 6. Also, for comparison, the result of low-temperature liquid-phase methanol synthesis using nickel fine powder and a Raney nickel-sodium methoxide composite catalytic system is shown as well.

Regarding nickel fine powder and a Raney nickel-sodium methoxide composite (soluble) catalytic system, high catalytic activity is shown both at low temperatures of 373 K and 353 K; whereas regarding a solid system using anion-exchange resin, only the consumption of carbon monoxide and the formation of methyl formate are seen, without the formation of methanol being confirmed.

TABLE 6
Liquid-phase Methanol Synthesis By Means Of Nickel - Ion-exchange
Resin (Or Sodium Methoxide) Composite Catalytic System

| | | CO | Methanol | Product [mmol] | | |
|---|---|---|---|---|---|---|
| | Temperature | Conversion | Selectivity | | Methyl | |
| Composite Catalyst | [K] | [%] | [%] | Methanol | Formate | Methane |
| Ni Powder - A26* | 373 | 66.0 | — | — | 35.2 | — |
| Ni Powder - A26 | 373 | 64.8 | — | — | 25.7 | — |
| Ni Powder - A26** | 373 | 58.7 | — | — | 43.7 | — |
| Ni Powder - A26 | 423 | 31.4 | — | — | 21.0 | — |
| Raney Ni - A26 | 423 | 30.1 | — | — | 25.6 | — |
| Raney Ni - TSA1200 | 393 | 53.5 | — | — | 11.5 | — |
| Ni Powder - NaOMe*** | 373 | 73.6 | 95.8 | 64.2 | 1.4 | — |
| Raney Ni - NaOMe¥ | 393 | 91.8 | 99.8 | 67.1 | trace | 0.1 |
| Raney Ni - NaOMe¥ | 353 | 71.3 | 94.2 | 55.0 | 1.7 | trace |

Reaction condition: 5.0 Mpa (H₂/CO/Ar=8/4/1), 2 h, Ni (powder or Raney) 1 g, resin A26 (or TSA1200) 20 ml, methanol 10 ml, triglyme 70 ml, (*A26:10 ml, *methanol 20 ml, **A26:40 ml, **triglyme 50 ml, ***NaOCH₃ 2.2 g (41 mmol), ***methanol 8.5 ml, ***triglyme 91.5 ml, Y E. S. Lee, K Aika, J Mol. Catal. A, 141, 241 (1999).

### REFERENCE EXAMPLE 4

### Catalytic Activity Behavioral Change in Reaction Gas Composition

Regarding a Raney copper-TSA1200 composite catalytic system, in order to examine catalytic activity behavioral change in reaction gas composition, reaction was executed at 393 K, with a system (a) in which a reaction gas was carbon monoxide/argon, and with a system (b) in which a reaction gas was hydrogen/argon and methyl formate was added to a reaction solution. Further, the reaction pressure is made to be equal to each partial pressure in methanol synthetic gas (5 MPa, H₂/CO/Ar=8/4/1). With the system (a) in which a reaction gas was carbon monoxide/argon, methanol was not generated, and a methyl formate formation amount of 28.1 mmol and a carbon monoxide conversion of 48.6% were obtained, which is thought to be caused by methanol carbonylation reaction. Further, decrease in pressure occurred only within 15 minutes or so after the start of the reaction, which is a temperature-rising process, and the pressure was constant thereafter. On the other hand, with the system (b) in which a reaction gas was hydrogen/argon and methyl formate was added to a reaction solution, 111.8 mmol of methanol and 37.6 mmol of carbon monoxide were generated, and the conversion of methyl formate was 80.2%. Further, regarding a temporal pressure change, the pressure rose in approximately 30 minutes after the start of the reaction, and after that, gradually decreased until the end of the reaction. Judging from the fact that carbon monoxide was generated, it is assumed that the pressure increase earlier in the reaction was caused by the formation of carbon monoxide due to decomposition reaction of methyl formate with ion-exchange resin as catalyst, and that the subsequent pressure decrease was caused by the consumption of hydrogen due to the progress of methyl formate hydrogenation decomposition reaction. According to the above results, it has been found out that carbonylation reaction of methanol is mainly caused by TSA1200, and that methyl formate formed is hydrogenated by Raney copper. In addition, it has been shown that regarding the latter, when there is no co-presence of CO, if hydrogen reacts, part thereof is decomposed into methanol and CO by TSA1200.

### REFERENCE EXAMPLE 5

### Low-Temperature Liquid-Phase Methanol Synthesis and Methyl Formate Hydrogenation Decomposition Reaction Using Raney Copper-Sodium Methoxide Composite (Soluble) Catalytic System

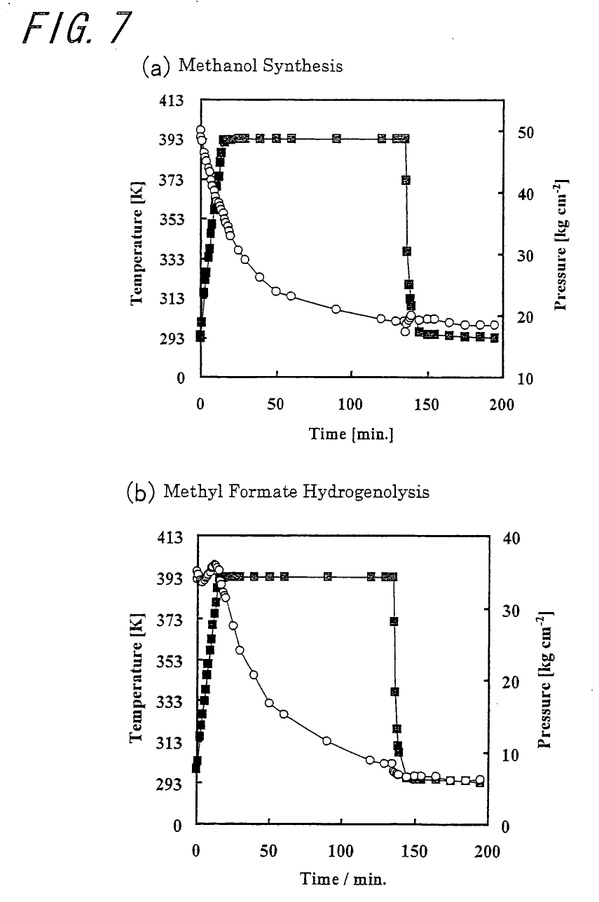

Table 7 shows the results of low-temperature liquid-phase methanol synthesis and methyl formate hydrogenation decomposition reaction using a Raney copper-sodium methoxide composite catalytic system. Also, temporal changes of pressure and temperature in a reaction were shown in FIG. 7. In the low-temperature liquid-phase methanol synthesis, a large pressure decrease was observed as soon as the reaction started, and an excellent catalytic characteristic of 91.4% in carbon monoxide conversion and 99.8% in methanol selectivity, was obtained.

In the methyl formate hydrogenation decomposition reaction, the pressure increased somewhat until 15 minutes after the start of the reaction, and afterward decreased dramatically. A methyl formate conversion of 98.8% and a methanol selectivity of 95.6% were obtained. Further, carbon monoxide was generated by 3.9 mmol. It is assumed that the pressure increase earlier in the reaction was caused by the formation of carbon monoxide due to decomposition reaction of methyl formate whose catalyst was sodium methoxide. As reported thus far, the methyl formate hydrogenation capability of a Raney copper-sodium methoxide system is great, and therefore the methanol synthesis capability thereof is great. However, the present system is nothing but soluble catalysts.

TABLE 7
Liquid-phase Methanol Synthesis Reaction At 393 K By Means Of Raney
Copper - sodium methoxide composite (soluble) catalytic system

| | | Methyl | | | | |
|---|---|---|---|---|---|---|
| | | Formate | Methanol | Product [mmol] | | |
| | CO | Conversion | Selectivity | | | Methyl |
| Reaction | Conversion | [%] | [%] | Methanol | CO | Formate |
| Methanol Synthesis | 91.4 | — | 99.8 | 66.3 | — | trace |
| Methyl Formate Hydrogenation | — | 98.8 | 95.6 | 84.3 | 3.9 | — |

Reaction condition: 393 K, 2 h, Raney copper 2 g, NaOCH₃ 2.2 g (41 mmol) Methanol synthesis: 5.0 Mpa (H₂/CO/Ar=8/4/1), methanol 30 ml, triglyme 70 ml.
Methyl formate hydrogenation: 3.5 Mpa (H₂/Ar=8/1), methyl formate 7.7 ml, methanol 22.3 ml, triglyme 70 ml.

1. A solid catalyst in which an ion-exchange resin anion-exchanged to be an alcoxide type is combined with a transition metal.

2. A solid catalyst according to claim 1, wherein copper or several kinds of metals including copper are combined as a transition metal/metals.

3. A solid catalyst according to claim 1, wherein an ion-exchange resin having allowable temperature range of 333 K to 453 K is combined.

4. A solid catalyst according to claim 1, wherein the mass ratio of an ion-exchange resin to a transition metal catalyst is between 1/100 and 100/1.

5. A solid catalyst according to claim 1, wherein the mass ratio of an ion-exchange resin to a transition metal catalyst is between 1/10 and 10/1.

6. A solid catalyst according to claim 1, which acts in a solution of methanol and a polar organic solvent mixed.

7. A solid catalyst according to claim 1, wherein an ion-exchange resin having a methoxide anion as an alcoxide to be exchanged is combined.

8. A reactor containing a solid catalyst in which an ion-exchange resin anion-exchanged to be an alcoxide type is combined with a transition metal.

9. A method of producing methanol, in which carbon monoxide and hydrogen are made to react in the presence of a solid catalyst in which an ion-exchange resin anion-exchanged to be an alcoxide type is combined with a transition metal.
